(19) United States
(12) Patent Application Publication
(10) Pub. No.: US 2026/0262926 A1
(43) Pub. Date: Sep. 10, 2026

(54) LOCKING TROCAR ENDOSCOPE CLEANER

(71) Applicants: Clark B. FOSTER, Houston, TX (US); Gerald Jay SANDERS, Houston, TX (US); Gerald FEUER, Houston, TX (US)

(72) Inventors: Clark B. FOSTER, Houston, TX (US); Gerald Jay SANDERS, Houston, TX (US); Gerald FEUER, Houston, TX (US)

(21) Appl. No.: 18/984,677

(22) PCT Filed: Jun. 16, 2023
(86) PCT No.: PCT/US2023/025574

§ 371 Date: Dec. 17, 2024

Related U.S. Application Data
(60) Provisional application No. 63/353,457, filed on Jun. 17, 2022.

(51) Int. Cl. A61B 1/12; A61B 1/00; A61B 17/34

(52) U.S. Cl. CPC A61B 1/126; A61B 1/00137; A61B 1/00154; A61B 17/3417; A61B 2017/3454

(57) ABSTRACT

Device adapted to be fitted to an open end of a trocar tube. Device includes a substantially cylindrical attachment tube operable to be coupled to the open end of the trocar tube. Device can also include a jaw extending away from the attachment tube and having a substantially conical distal end located away from the attachment tube. Jaw has sidewalls corresponding to a portion of the circumference of the tube. Device also can include a sheath extending away from the attachment tube and having a distal end that extends toward the substantially conical distal end of the jaw. Sheath has sidewalls corresponding to another portion of the circumference of the tube and the sidewalls of the sheath and the sidewalls of the jaw being biased towards each other. Device can include at least one locking mechanism operable to lock the jaw in a closed configuration.

### FIELD

The present disclosure relates generally to a lens cleaner for an endoscope. In at least one example, the present disclosure relates to an assembly to be coupled to a trocar and operable to clean an endoscope.

### BRIEF DESCRIPTION OF THE DRAWINGS

Implementations of the present technology will now be described, by way of example only, with reference to the attached figures, wherein:

FIG. 1A illustrates an example of a jaw lock assembly operable to lock jaws of a trocar for allowing an endoscope to pass therethrough, according to at least one instance of the present disclosure.

FIG. 1B illustrates an example of a jaw lock assembly in an open configuration, according to at least one instance of the present disclosure.

FIG. 2 illustrates another example of a jaw locking assembly, according to at least one instance of the present disclosure.

FIG. 3A illustrates an example wherein a jaw and slider spring are illustrated in a locked or closed configuration, according to at least one instance of the present disclosure.

FIG. 3B illustrates an example wherein a jaw and slider spring are illustrated in a partially open or released configuration, according to at least one instance of the present disclosure.

FIG. 4 illustrates an example of slider and jaw locking assembly, according to at least one instance of the present disclosure.

FIGS. 5A and 5B illustrates an example of a camera clamp operable to hold the camera in position relative to the trocar during insertion of the trocar into a patient, according to at least one instance of the present disclosure.

FIG. 6 illustrates an example of a trocar, camera, and collar clamp during the insertion into a patient, according to at least one instance of the present disclosure.

FIG. 7 illustrates an example of a jaw lock assembly and flexible spring attachment, according to at least one instance of the present disclosure.

FIG. 8 illustrates an assembly providing for a mechanism to hold the flexible spring in place, according to at least one instance of the present disclosure.

FIG. 9 illustrates another example of the jaw lock assembly in a partially extended configuration, according to at least one instance of the present disclosure.

FIG. 10 illustrates another example of a biasing member withing a receiving portion, according to at least one instance of the present disclosure.

FIG. 11 illustrates an attachment device for shipping a device and holding a camera in place relative to the device, according to at least one instance of the present disclosure.

### DETAILED DESCRIPTION

It will be appreciated that for simplicity and clarity of illustration, where appropriate, reference numerals have been repeated among the different figures to indicate corresponding or analogous elements. In addition, while specific details are set forth in order to provide an understanding of the examples described herein, it will be appreciated by those skilled in the art that the examples described herein can be practiced without these specific details. In other instances, methods, procedures and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

Several definitions that apply throughout this disclosure will now be presented. The term “coupled” is defined as connected, whether directly or indirectly through intervening components, and is not necessarily limited to physical connections. The term “communicatively coupled” is defined as connected, either directly or indirectly through intervening components, and the connections are not necessarily limited to physical connections, but are connections that accommodate the transfer of data between the so-described components. The connection can be such that the objects are permanently connected or releasably connected. The term “outside” refers to a region that is beyond the outermost confines of a physical object. The terms “comprising,” “including” and “having” are used interchangeably in this disclosure. The terms “comprising,” “including” and “having” mean to include, but not necessarily be limited to the things so described. The term “substantially” is defined to be essentially conforming to the particular dimension, shape or other word that substantially modifies, such that the component need not be exact.

In the field of laparoscopic or other minimally invasive surgery, the surgeon relies on a clear view of the surgical site within the abdominal cavity of the patient. The view is provided by a specialized cylindrical endoscopic or laparoscopic camera that passes through a tubular trocar. The trocar is placed in the abdominal wall of the patient to provide a pathway from outside of the patent into the abdominal cavity. The trocar also provides a pathway where the cylindrical camera can be inserted rotated and manipulated as required to view the surgical site.

The present disclosure can be used with both manual operations and robotic operations. When implemented with either the robotic or manual operations, the improved cleaning offered for the camera results in less downtime, less confusion on the operating floor, and allows the surgeon to keep up the momentum in achieving a quick surgery because the camera does not have to be removed from the trocar tube.

The present disclosure provides an improved version of a camera cleaning apparatus. The present disclosure has a several features that improvements over existing devices. One of the improvements is the ability to lock the jaw relative to a sheath. The ability to lock the jaw to the sheath allows for the device to be inserted into a patient and prevent the jaw from separating from the sheath. Additionally, the device can be operated simply by inserting the camera through the trocar tube and passing through the device. The camera can unlock the device and additionally operate the wiper blade to remove any liquids, films, or objects on the camera. Furthermore, the device features a biasing member that is concealed to prevent tissue from hanging on the biasing member or mounting structure.

The device includes a substantially cylindrical attachment tube that is operable to be press fit onto a distal end of a trocar tube. The device can be manufactured in a variety of different sizes and diameters according to the desired trocar tube. The substantially cylindrical attachment tube can be operably configured for a desired trocar tube. The device can also include a hinge connecting the substantially cylindrical attachment tube to a jaw. The jaw can be operable to swing laterally to provide a path for the camera to pass therethrough. While the present disclosure describes a device that can be installed onto an existing trocar tube, the present technology can be formed as part of a trocar rather than being a separate member. In at least one example, the device and/or trocar can be formed of substantially transparent or translucent material to allow a camera to capture images therethrough.

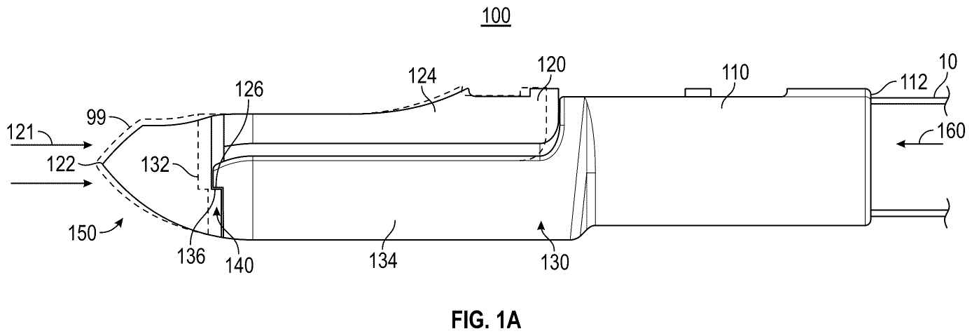

FIG. 1A illustrates an example of a jaw lock assembly operable to lock a jaw to a sheath of a device that is operable for allowing an endoscope to pass therethrough. As illustrated, a device 100 adapted to be fitted to an open end 20 of a trocar tube 10. The device 100 can include a substantially cylindrical attachment tube 110 operable to be coupled to the open end of the trocar tube 10. The device 100 can also include a jaw 120 extending away from the attachment tube 110 and having a substantially conical distal end 122 located away from the attachment tube 110, wherein the jaw 120 has sidewalls 124 corresponding to a portion of the circumference of the attachment tube 110. The device 100 can also include a sheath 130 extending away from the attachment tube 110 and having a distal end 132 that extends toward the substantially conical distal end 122 of the jaw 120, wherein the sheath 130 has sidewalls 134 corresponding to another portion of the circumference of the attachment tube 110 and the sidewalls 134 of the sheath 130 and the sidewalls 124 of the jaw 120 being biased towards each other. The device 100 can further include at least one locking mechanism 140 operable to lock the jaw 120 in a closed configuration 150. The at least one locking mechanism 140 can include a jaw locking tab 136 and a jaw engagement lock portion 126. As illustrated the jaw locking tab 136 includes a substantially flat portion that is operable to slide past a substantially flat portion of the jaw engagement portion 126. The two substantially flat portions prevent the jaw 120 from pivoting relative to the sheath 130. Once the camera contacts a wiper blade and extends the jaw 120 relative to the sheath 130, the substantially flat portion of the jaw locking tab 136 extends beyond the substantially flat portion of the jaw engagement portion 126, so that the jaw 120 can pivot relative to the sheath 130.

FIG. 1B illustrates the jaw lock assembly of FIG. 1 in an open configuration. As illustrated, the jaw 120 has pivoted relative to the sheath 130 such that an angle 123 is formed between the sheath 130 and the jaw 120.

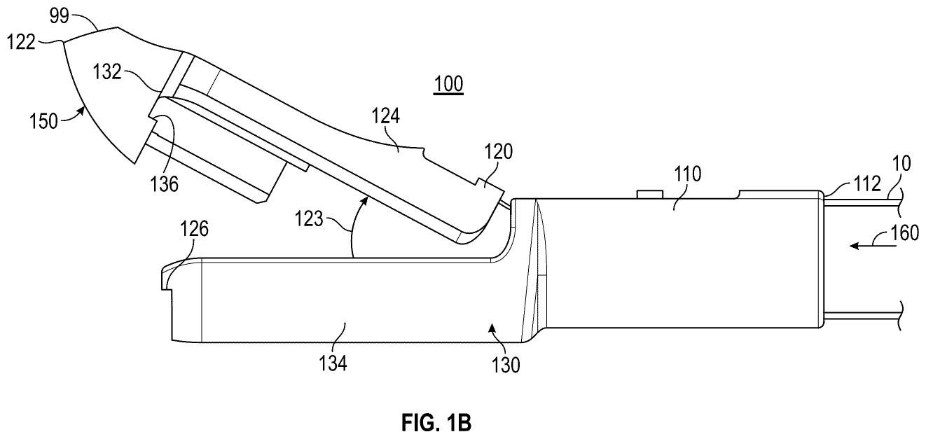

FIG. 2 illustrates another example of a jaw locking assembly 140. The locking assembly can move in a locked direction 141 or an unlock direction 143. The motion of the jaw locking assembly 140 is controlled by insertion of the camera through the device 100. The device can be configured to have a range of travel 142. The range of travel 142 allows for the engagement mechanism 144 to move and release the jaw 120.

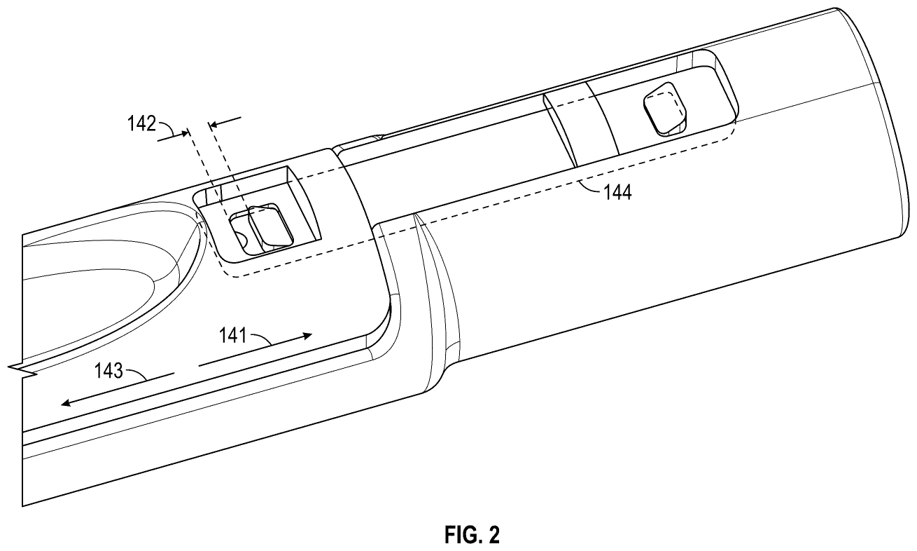

FIG. 3 illustrates an example device 100 wherein a jaw 120 and slider spring 183 are illustrated. The slider 180 can sweep across the centerline 302 of a lens of the camera 300. The slider 180 comprises a wiper blade 182 that is operable to sweep, in a single continuous motion, across the centerline 302 of a lens of a camera.

The slider spring 183 biases the slider 180 towards the camera 300 so that the wiper 182 resists motion of the camera 300 in the axial direction such as the centerline 302 of the camera as shown. The bottom edge 181 of the wiper 182 contacts the camera 300 first. The wiper 182 can be of a different material from the slider 180. This allows the wiper 182 to be a material that is designed to remove the fluids and/or substances from the camera 300. The wiper 182 can be operable to be engaged by a camera 300 traversing through the attachment tube 110, whereby the wiper blade 182 crosses the centerline 302 and allows the camera 300 to pass beyond the wiper 182. The wiper blade 182 can be biased towards the camera 300 by an elastic slider spring 183. The wiper blade 182 has a lower edge 181 that extends away from the jaw 120 and extends beyond a centerline 302 with respect to the attachment tube 110.

The slider 180 moves in an axial direction along with the wiper 182 until the camera contacts a cam 200. The cam 200 operates to open the jaw. The cam 200 can be integrally formed with the jaw 120. The cam 200 can face the open end of the trocar tube 10. The cam 200 as illustrated is in a linear configuration. This allows for a smooth opening of the jaw 120. The jaw is operable to be moved to a released configuration upon contact of a camera 300 with the cam 200. Once the cam 200 is contacted, the jaw 120 slides in a distal direction thereby releasing the jaw locking tab 136 from jaw engagement portion 126. Once the jaw locking tab 136 is released, the jaw 120 is operable to pivot upward and away from sheath 130. In other examples, the jaw 120 can pivot such that a distal end of the jaw pivots radially away from a centerline of the sheath. In the illustrated example of FIG. 3, the attachment tube 110 is not coupled with a trocar tube for clarity of illustration.

FIG. 3B illustrates an example wherein a jaw 120 and slider spring 183 are illustrated in a partially open or released configuration, according to at least one instance of the present disclosure. The position as illustrated in FIG. 3B is not a fully open configuration as the slider spring 183 is not fully compressed and the camera 300 has not been fully extended beyond the jaw 120. As illustrated the centerline 305 of the jaw 120 forms an angle α with the centerline 302 of the camera. Additionally, the cam 200 is in contact with the camera 300. The wiper 182 has slid past the centerline 302 of the camera 300 thereby wiping a lens on a front face of the camera 300. As the camera 300 continues through and exits the jaw 120, the wiper will rise higher and the slider spring 183 will compress further.

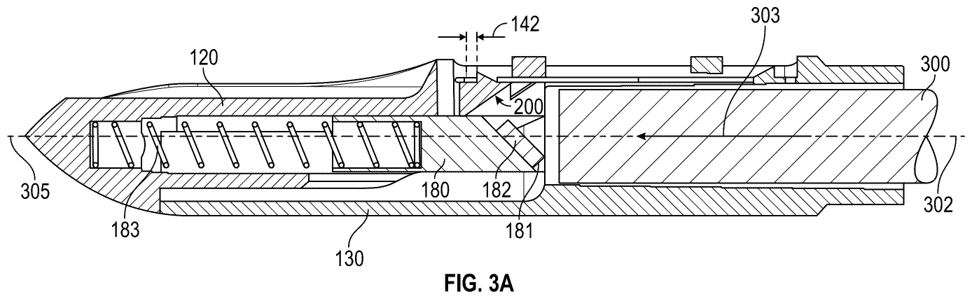

FIG. 4 illustrates an example of slider 180 and jaw locking assembly 140 in a partial view for clarity. FIG. 4 illustrates that the sheath 130 can include at least one jaw locking tab 136 that is a pair of jaw locking tabs having a first jaw locking tab 135 and a second jaw locking tab 137. The slider 180 is illustrated as well. Further illustrated are a post 188 and an aperture 187 formed in a bar 186. The post can include a cap 189 that is designed to be inserted through the aperture 187.

As illustrate in FIGS. 1-4, the device 100 can include a jaw 120 includes at least one jaw locking tab 136 that extends from the sheath 130 at the distal end 132. The jaw 120 can include at least one a jaw engagement lock portion 126 that is operable to receive the at least one jaw locking tab 136, whereby the jaw 120, in the closed configuration, is prevented from opening relative to the sheath 130. The at least one jaw locking tab 136 comprises a first jaw locking tab 135 on an upper side 131 of the sheath 130 and a second jaw locking tab 137 on an opposite upper side 133 of the sheath 130. The camera 300 is operable to move the jaw in an axial direction away from the sheath 130 and the attachment tube 110, such that upon a jaw engagement lock portion 126 extending beyond the at least one jaw locking tab 136, the jaw is in a released configuration denoted by dotted line 99. Upon subsequent movement of the camera 300 in an axial direction moves the jaw 120 away from the sheath 130 and pivoting a distal end 122 of the jaw 120 back towards the substantially cylindrical attachment tube 110. The jaw 120 can be released from the closed configuration by sliding in an axial direction.

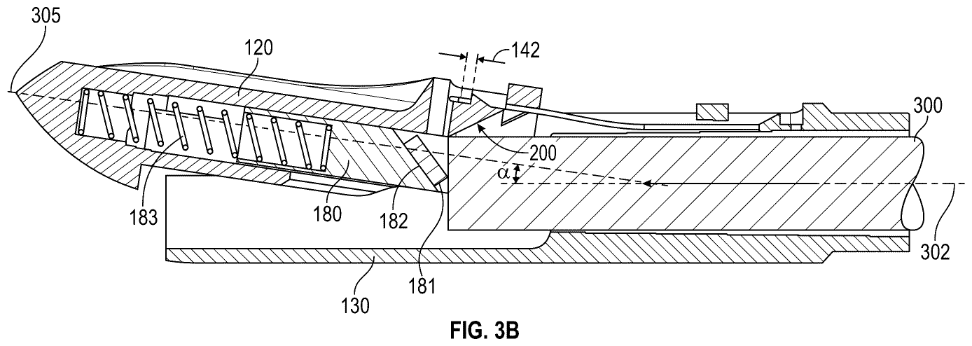

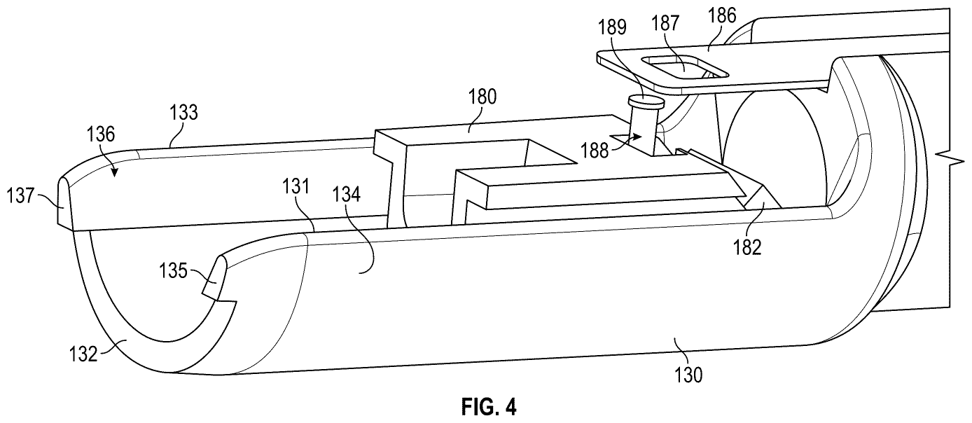

FIGS. 5A and 5B illustrates an example of a camera clamp operable to hold the camera (not shown—but further illustrated in FIG. 6) in position relative to the trocar during insertion of the trocar into a patient. The camera clamp 500 can include a pair of handles 502. The handles 502 can be moved from a closed position 501 to an open position 599. The camera clamp 500 includes a camera engagement portion 508 that forms a through hole 506 in a closed configuration. The camera engagement portion 508 can be soft but firm. Additionally, the camera clamp can include a locking portion 504 that is operable to lock the clamp to the camera. The locking portion 504 can be in the form of a ratcheting lock. The handles 502 can pivot about a pivot connection 510. The pivot connection 510 can be a rod or a shaft.

FIG. 6 illustrates an example of a trocar 30, camera 300, and camera clamp 500 during the insertion into a patient, according to at least one instance of the present disclosure. AS illustrated the device 100 can be operable to be inserted into the patient on the end of the trocar 30. The device 100 is coupled to the trocar tube 10. The cameraclamp 500 prevents the camera 300 from opening the device 100 as described above. In at least one example, the device 100 can be configured to be see through so that the operator can see where the device 100 is inside the patient during the insertion process.

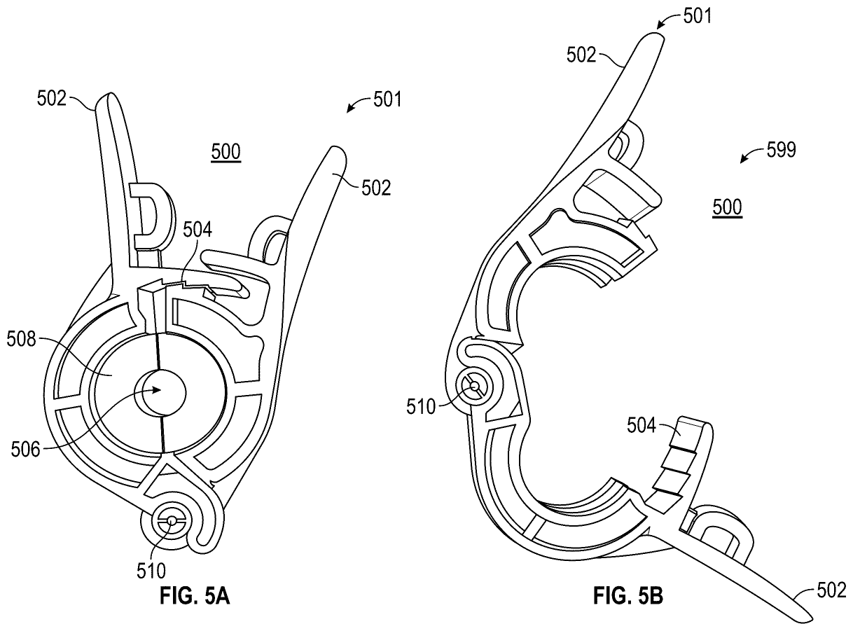

FIG. 7 illustrates an example of a biasing member 410 coupled at a first end 412 to the jaw 120 and coupled at a second end 414, opposite the first end 412, to the substantially cylindrical attachment tube 110. The biasing member 410 is operable to push the jaw 120 towards the sheath 130. The biasing member 410 has mounting portions 420 that allow attachment members 424 to pass therethrough and secure the biasing member 410 to the jaw 120 and the substantially cylindrical attachment tube 110. The jaw 120 forms a first biasing member receiving portion 430 that forms a first recess 432 configured to receive a portion 416 of the biasing member 410. The substantially cylindrical attachment tube 110 forms a second biasing member receiving portion 440 that forms a second recess 442 for receiving another portion 418 of the biasing member 410. Additionally, the set of mounting portions 420 that are located closest to the attachment tube 110 can be formed as slots 450. The slots 450 allow the biasing member 410 to slide relative to the proximal mounting member 452. The proximal mounting members 452 can be further coupled together through a wire 462. The wire 462 provides enhanced security of the proximal mounting members 452. Additionally it holds biasing member 410 in place. The slots 450 allow for axial motion in a distal direction and proximal direction. The jaw 120 moves in the distal direction as the camera is extended therethrough. The motion in the proximal direction occurs as the distal end 122 penetrates the patient.

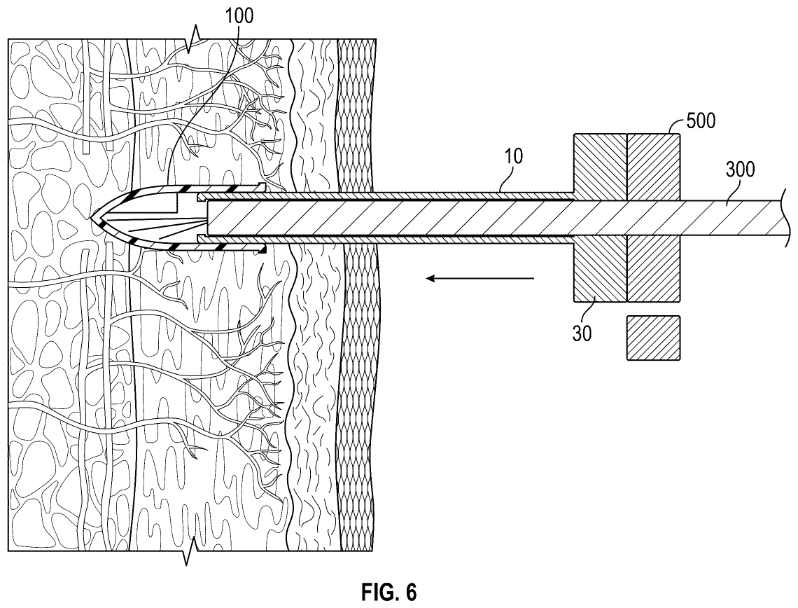

FIG. 8 illustrates an assembly providing for a mechanism to hold the flexible spring in place, according to at least one instance of the present disclosure. As illustrated the walls 441 of the second biasing member receiving portion 440 also provide for a way to hold the biasing member 410 in place to prevent lateral movement of the biasing member 410. The first recess 432 and second recess 442 are formed within walls 441 that extend beyond the height 50 of the biasing member 410 and attachment members 424. The device 100 can also include an elastic member 470 covering the first biasing member receiving portion 430. In at least one example, the elastic member 470 further covers the second biasing member receiving portion 440. The elastic member 470 is shown as being substantially even with the walls 441 so as to provide a smooth transition. Additionally, the walls 441 can include a chamfer to provide a smooth transition. In other examples, the elastic member 470 may be below the walls. When the elastic member 470 is above the walls, the edges will not be as large as shown as it is a thin wall member. Rather the proportions are larger for illustration purposes. In some examples, the height 50 of the biasing member 410 and attachment members 424 extend beyond the walls, the elastic member 470 can provide a smoothing surface to prevent the protrusions of the biasing member 470 and attachment member 424 from catching on tissue. Furthermore, the elastic member 470 can be implemented in configurations where there are no walls. Additionally, the elastic member 470 can be configured to have a predetermined amount of compliance.

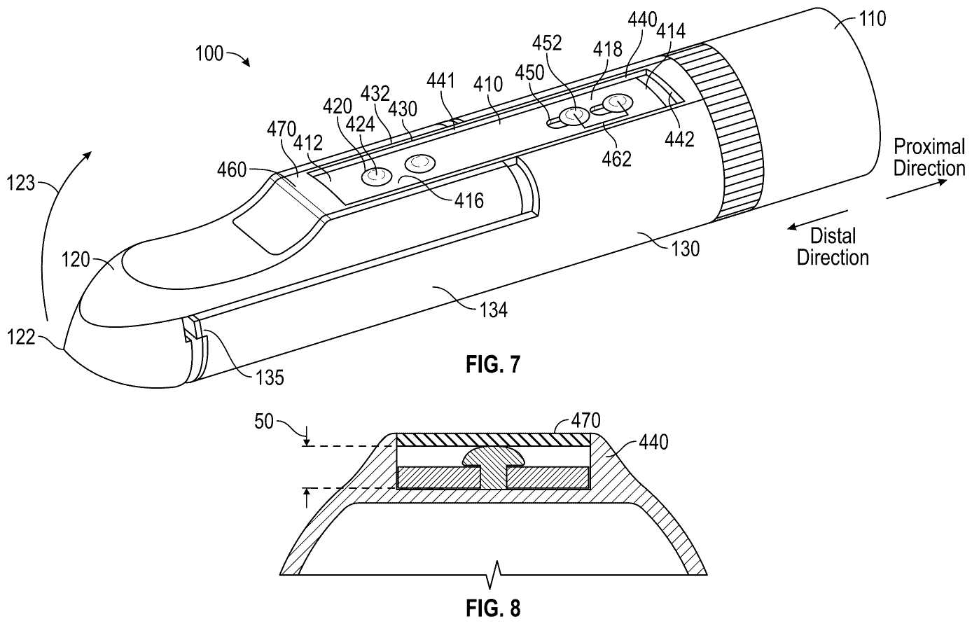

FIG. 9 illustrates an example of a biasing member 410 coupled at a first end 412 to the jaw 120 and coupled at a second end 414, opposite the first end 412, to the substantially cylindrical attachment tube 110. The first biasing member receiving portion 430 includes a penetrating edge 460 that is nearest to the distal end 122 of the jaw 120 and the penetrating edge 460 includes rounded edges to allow tissue to easily pass over the first biasing member receiving portion 430. In this example the other features can be substantially the same as illustrated above in regards to FIG. 7. As illustrated the device 100 is shown in a clear view so that the slider spring 183 is visible.

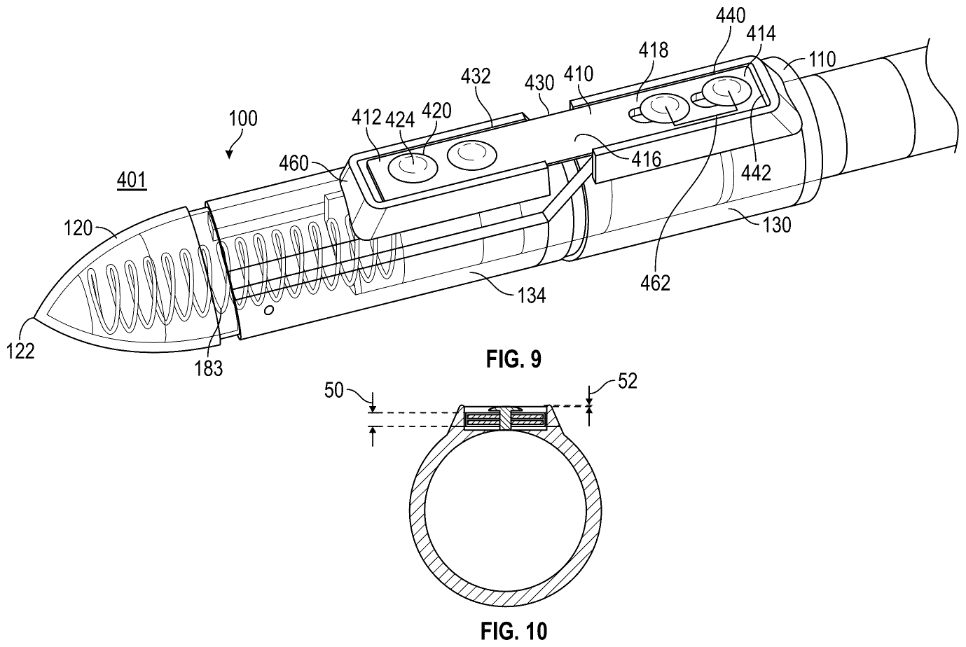

FIG. 10 illustrates another example of a biasing member 410 withing a receiving portion. As illustrated the biasing member 410 and attachment member 424 have a height of 50 that is a distance 52 from the top edge of wall 441. This allows the tissue to pass over the top of the device without catching on the biasing member 410 and attachment member 424.

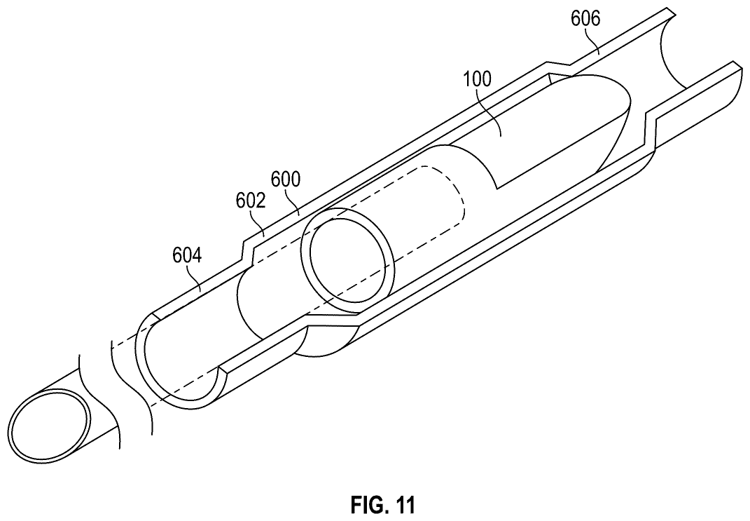

FIG. 11 illustrates an attachment device 600 for shipping a device 100 and holding a camera 300 in place relative to the device 100. The attachment device can have a wider end 602 that allows the device 100 to be inserted therein. A narrower end 606 opposite the wider end 602 prevents the device from sliding therethrough and holds the device in place. A trocar receiving tube portion 604 allows the trocar tube to be inserted into the device so that the tip of the device does not contact an operator. Furthermore, the attachment device 600 allows for the device to be more easily coupled to the trocar tube.

The embodiments shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the embodiments described above may be modified within the scope of the appended claims.

Illustrative examples of the disclosure include:

Aspect 1. A device adapted to be fitted to an open end of a trocar tube, the device comprising: a substantially cylindrical attachment tube operable to be coupled to the open end of the trocar tube; a jaw extending away from the attachment tube and having a substantially conical distal end located away from the attachment tube, wherein the jaw has sidewalls corresponding to a portion of the circumference of the tube; a sheath extending away from the attachment tube and having a distal end that extends toward the substantially conical distal end of the jaw, wherein the sheath has sidewalls corresponding to another portion of the circumference of the tube and the sidewalls of the sheath and the sidewalls of the jaw being biased towards each other; at least one locking mechanism operable to lock the jaw in a closed configuration.

Aspect 2. The device as recited in Aspect 1, wherein the jaw is operable to be released from the closed configuration by sliding in an axial direction.

Aspect 3. The device as recited in any of Aspects 1 to 2, wherein the jaw includes a cam facing the open end of the trocar tube.

Aspect 4. The device of any of Aspects 1 to 3, wherein the cam is integrally formed with the jaw, and the jaw is operable to be moved to a released configuration upon contact of a camera with the cam.

Aspect 5. The device as recited in any of Aspects 1 to 4, wherein the jaw includes at least one jaw locking tab that extends from the sheath at the distal end.

Aspect 6. The device as recited in any of Aspects 1 to 5, wherein the at least one jaw locking tab comprises a first jaw locking tab on an upper side of the sheath and a second jaw locking tab on an opposite upper side of the sheath.

Aspect 7. The device as recited in any of Aspects 1 to 6, wherein the camera is operable to move the jaw in an axial direction away from the sheath and the attachment tube, such that upon a jaw engagement portion extending beyond the at least one jaw locking tab, the jaw is in a released configuration.

Aspect 8. The device as recited in any of Aspects 1 to 7, wherein upon subsequent movement of the camera in an axial direction moves the jaw away from the sheath and pivoting a distal end of the jaw radially away from a centerline of the sheath.

Aspect 9. The device as recited in any of Aspects 1 to 8, wherein the jaw includes at least one a jaw engagement lock portion that is operable to receive the at least one jaw locking tab, whereby the jaw, in the closed configuration, is prevented from opening relative to the sheath.

Aspect 10. The device as recited in any of Aspects 1 to 9, further comprising a slider operable to sweep across a centerline of a lens of a camera.

Aspect 11. The device as recited in any of Aspects 1 to 10, wherein the slider comprises a wiper blade that is operable to sweep, in a single continuous motion, across the centerline of a lens of a camera.

Aspect 12. The device as recited in any of Aspects 1 to 11, further comprising: a wiper blade coupled to the slider, wherein the wiper blade has a lower edge that extends away from the jaw and extends beyond a centerline with respect to the attachment tube; wherein the wiper blade is operable to be engaged by a camera traversing through the attachment tube, whereby the wiper blade crosses the centerline and allows the camera to pass beyond the wiper, which is operable to remove fluids and/or substances from the camera.

Aspect 13. The device as recited in any of Aspects 1 to 12, further comprising: a biasing member coupled at a first end to the jaw and coupled at a second end, opposite the first end, to the substantially cylindrical attachment tube, wherein the biasing member is operable to push the jaw towards the sheath.

Aspect 14. The device as recited in any of Aspects 1 to 13, wherein the biasing member has mounting portions that allow mounting members to pass therethrough and secure the biasing member to the jaw and the substantially cylindrical attachment tube.

Aspect 15. The device as recited in any of Aspects 1 to 14, wherein the jaw forms a first biasing member receiving portion that forms a first recess configured to receive a portion of the biasing member.

Aspect 16. The device as recited in any of Aspects 1 to 15, wherein the substantially cylindrical attachment tube forms a second biasing member receiving portion that forms a second recess for receiving another portion of the biasing member.

Aspect 17. The device as recited in any of Aspects 1 to 16, wherein the first recess and second recess are formed within walls that extend beyond a height of the biasing member and attachment members.

Aspect 18. The device as recited in any of Aspects 1 to 17, wherein the first biasing member receiving portion includes a penetrating edge that is nearest to the distal end of the jaw and the penetrating edge includes rounded edges to allow tissue to easily pass over the first biasing member receiving portion.

Aspect 19. The device as recited in any of Aspects 1 to 18, further comprising an elastic member covering the first biasing member receiving portion.

Aspect 20. The device as recited in any of Aspects 1 to 19, wherein the elastic member further covers the second biasing member receiving portion.

What is claimed is:

1. A device adapted to be fitted to an open end of a trocar tube, the device comprising:
a substantially cylindrical attachment tube operable to be coupled to the open end of the trocar tube;
a jaw extending away from the attachment tube and having a substantially conical distal end located away from the attachment tube, wherein the jaw has sidewalls corresponding to a portion of the circumference of the tube;
a sheath extending away from the attachment tube and having a distal end that extends toward the substantially conical distal end of the jaw, wherein the sheath has sidewalls corresponding to another portion of the circumference of the tube and the sidewalls of the sheath and the sidewalls of the jaw being biased towards each other;
at least one locking mechanism operable to lock the jaw in a closed configuration.

2. The device as recited in claim 1, wherein the jaw is operable to be released from the closed configuration by sliding in an axial direction.

3. The device as recited in claim 1, wherein the jaw includes a cam facing the open end of the trocar tube.

4. The device of claim 3, wherein the cam is integrally formed with the jaw, and the jaw is operable to be moved to a released configuration upon contact of a camera with the cam.

5. The device as recited in claim 4, wherein the jaw includes at least one jaw locking tab that extends from the sheath at the distal end.

6. The device as recited in claim 5, wherein the at least one jaw locking tab comprises a first jaw locking tab on an upper side of the sheath and a second jaw locking tab on an opposite upper side of the sheath.

7. The device as recited in claim 5, wherein the camera is operable to move the jaw in an axial direction away from the sheath and the attachment tube, such that upon a jaw engagement portion extending beyond the at least one jaw locking tab, the jaw is in a released configuration.

8. The device as recited in claim 7, wherein upon subsequent movement of the camera in an axial direction moves the jaw away from the sheath and pivoting a distal end of the jaw radially away from a centerline of the sheath.

9. The device as recited in claim 5, wherein the jaw includes at least one a jaw engagement lock portion that is operable to receive the at least one jaw locking tab, whereby the jaw, in the closed configuration, is prevented from opening relative to the sheath.

10. The device as recited in claim 1, further comprising a slider operable to sweep across a centerline of a lens of a camera.

11. The device as recited in claim 10, wherein the slider comprises a wiper blade that is operable to sweep, in a single continuous motion, across the centerline of a lens of a camera.

12. The device as recited in claim 10, further comprising:
a wiper blade coupled to the slider, wherein the wiper blade has a lower edge that extends away from the jaw and extends beyond a centerline with respect to the attachment tube;
wherein the wiper blade is operable to be engaged by a camera traversing through the attachment tube, whereby the wiper blade crosses the centerline and allows the camera to pass beyond the wiper, which is operable to remove fluids and/or substances from the camera.

13. The device as recited in claim 1, further comprising:
a biasing member coupled at a first end to the jaw and coupled at a second end, opposite the first end, to the substantially cylindrical attachment tube, wherein the biasing member is operable to push the jaw towards the sheath.

14. The device as recited in claim 13, wherein the biasing member has mounting portions that allow mounting members to pass therethrough and secure the biasing member to the jaw and the substantially cylindrical attachment tube.

15. The device as recited in claim 13, wherein the jaw forms a first biasing member receiving portion that forms a first recess configured to receive a portion of the biasing member.

16. The device as recited in claim 15, wherein the substantially cylindrical attachment tube forms a second biasing member receiving portion that forms a second recess for receiving another portion of the biasing member.

17. The device as recited in claim 16, wherein the first recess and second recess are formed within walls that extend beyond a height of the biasing member and attachment members.

18. The device as recited in claim 17, wherein the first biasing member receiving portion includes a penetrating edge that is nearest to the distal end of the jaw and the penetrating edge includes rounded edges to allow tissue to easily pass over the first biasing member receiving portion.

19. The device as recited in claim 18, further comprising an elastic member covering the first biasing member receiving portion.

20. The device as recited in claim 19, wherein the elastic member further covers the second biasing member receiving portion.
